(19) United States
(12) Patent Application Publication
(10) Pub. No.: US 2003/0023139 A1
(43) Pub. Date: Jan. 30, 2003

(54) Clitoris stimulator device

(76) Inventor: Janet J. Hartz, Mercerville, NJ (US)

Correspondence Address: Kenneth Watov, Esq., WATOV & KIPNES, P.C., P.O. Box 247, Princeton Junction, NJ, 08550, US

(21) Appl. No.: 09/915,724

(22) Filed: Jul. 27, 2001

(51) Int. Cl. A61F 5/00

(52) U.S. Cl. 600/38

(57) ABSTRACT

The present invention is generally directed to a clitoris stimulating device which comprises a semi-rigid elongate member including a plurality of ridges extending radially therearound in abutting, successive arrangement with one another, the elongate member further includes a rigid handle affixed to one end thereof, and a vibrator device in operative engagement with the elongate member.

### FIELD OF THE INVENTION

The present invention relates generally to a device for providing sexual stimulation, and more particularly, to a device for enhancing a woman's sexual activity and pleasure through stimulation of the clitoris.

### BACKGROUND OF THE INVENTION

Various known devices for stimulating the female genitalia have been developed for the purpose of providing sexual gratification and climax during sexual intercourse, foreplay, and masturbation. Generally, they are used for personal pleasure and recreation by providing the user healthy sexual outlets. They are sometimes used in combination with sex-therapy programs. The sexual devices can provide the user an opportunity for sexual exploration and development, alleviate fears and anxiety about sexual performance, aid in the development of one's sensual capacities, and even facilitate verbal, emotional and sexual communication between sex partners. The most common form of the devices is the vibrator, which operates by applying pressure, friction, and vibration to the vagina and/or external areas of the genitalia.

Many of the known sexual devices available on the market are comprised of multiple parts and are sometimes prone to mechanical failure after several uses. Devices comprised of multiple parts are also more difficult and costlier to manufacture and fabricate. In addition, some devices are difficult to use and require some level of physical dexterity which disrupts or diminishes the sexual mood of the user and/or partner. Many devices particularly those imparting motion require a power source for operation which may likewise fail.

For the foregoing reasons, there is a need for a sexual stimulating device which is simple and easy to use and operate while being effective for creating sexual arousal in females quickly and successfully. There is a further need for a discreet sexual stimulating device configured for effectuating stimulating contact with the clitoris of a woman and which may be used for assisting the woman in achieving an orgasm quickly and easily. The device of the present invention may be operated manually or in combination with a vibrator for providing vibratory sensation to an erogenous zone of the user to increase sexual stimulation and arousal. There is also a need for a sexual stimulating device which can be manufactured in a cost efficient manner.

### SUMMARY OF THE INVENTION

The present invention provides a clitoris stimulating device which provides the user sexual stimulation and arousal. The device is adapted to pleasurably contact a woman's external genitalia areas including the clitoris for enhancing sexual activity and providing sexual stimulation. The device may be fabricated in a cost effective and simple manner. The device is capable of providing mechanical stimulation to the external genitalia of a woman by manual operation of the user or in cooperation with a vibrator device. The device is simple and easy to use and operate requiring little instruction.

There is provided for one aspect of the present invention, a clitoris stimulating device which comprises an elongate member including proximal and distal ends, and a plurality of corrugations extending in abutting arrangement therebetween.

### BRIEF DESCRIPTION OF THE DRAWINGS

Various embodiments of the invention are described in detail below with reference to the drawings, in which like items are identified by the same reference designation, wherein:

FIG. 1 is a perspective view of a clitoris stimulating device according to one embodiment of the present invention;

FIG. 2 is a bottom perspective view of the clitoris stimulating device according to the present invention being used for stimulating a clitoris along its side;

FIG. 3 is a perspective assembly view of a second embodiment of the clitoris stimulating device adapted for attachment with a vibrator device;

FIG. 4 is a perspective view of a third embodiment of the clitoris stimulating device;

FIG. 5 is a perspective view of a fourth embodiment of the clitoris stimulating device; and

FIG. 6 is a perspective view of a fifth embodiment of the clitoris stimulating device.

### DETAILED DESCRIPTION OF THE INVENTION

The present invention is generally directed to a clitoris stimulating device designed for providing a pleasurable sexual sensation to a woman through direct stimulation of the female clitoris and surrounding external areas of the female genitalia. The clitoris stimulating device provides a heightened stimulating sensation while being very easy, safe and comfortable to use. In addition, the clitoris stimulating device is adapted for heightening and stimulating orgasms quickly and successfully. The clitoris stimulating device may be used for self-stimulation or on a partner, and is very simple and inexpensive to fabricate. The clitoris stimulating device provides mechanical stimulation to the external genitalia of a female to supplement and enhance sexual activity, and to assist a woman who may be incurring difficulties in achieving orgasms. The clitoris stimulating device may be further adapted to be used in combination with a vibrator device for providing further heightened sexual sensation.

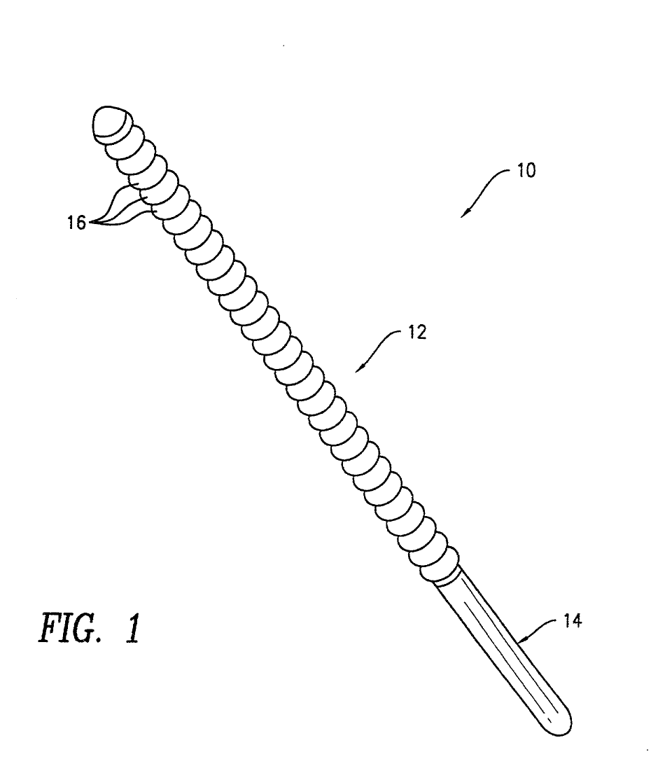

Referring now to the drawings, FIG. 1 shows a clitoris stimulating device 10 for one embodiment of the present invention. The device 10 is an elongate member comprised of an active contact portion 12 and a handle 14. The device 10 is composed of a resilient, semi-rigid material which provides a degree of flexibility for permitting a range of bending movement with a level of stiffness for imparting a pleasurable tactile contact with the erogenous zones of the female genitalia. The resilient, semi-rigid material includes, but is not limited to, plastic polymers, elastomers, and similar materials. Preferably, the material is also hypoallergenic. The handle 14 is generally rigid for providing the user with control of the device 10 during use as will be described. The handle 14 permits the user to manipulate and adjust the angle and motion of the device 10 for attaining an optimal level of sexual stimulation and sensation, preferably, using one hand.

The active contact portion 12 is composed of a plurality of corrugations or ridges 16 which may include a regular or irregular pattern. The plurality of corrugations 16 comprise repetitive areas of alternating peaks and valleys each of which may comprise any size, shape, pattern, configuration, texture, dimension, and the like. The end of the of the active portion 12 opposite from the handle 14 is preferably rounded forming a bulbous head to avoid inflicting injury to the body. Movement of the corrugations 16 across the areas of the user's genitalia especially the clitoral region, provides the pleasurable sensations as they touch and slip thereacross. The degree of stiffness or flexibility of the corrugations 16 may also be adjusted according to the user's tastes and preferences. It is further preferable that the surface of the active contact portion 12 retains a low coefficient of friction. As an alternative, a lubricating agent may be applied to the surface of the active contact portion 12 or to the female genitalia prior to use as will be described. In a preferred embodiment of the invention, the contact portion 12 is configured as successively stacked rounded bands 16, as shown.

For illustrating purposes only, the length of the active contact portion 12 is about 10 to 11 inches, and the diameter is 0.75 to 1 inch, although other dimensions could be used without departing from the scope of the invention.

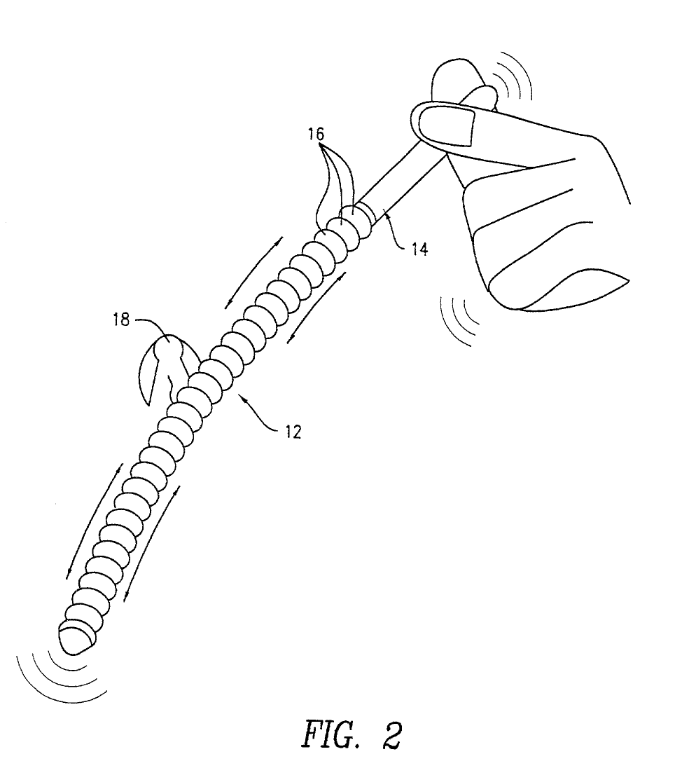

Referring to FIG. 2, the device 10 is shown in contact with a clitoris 18 of a user or a partner for initiating stimulation. The user holds the handle 14 for drawing the device 10 in a back and forth motion against the clitoris 18 and surrounding areas in order to desirably induce sexual arousal, and eventually, orgasm. In this manner, the active contact portion 12 rubs against the clitoris 18. The device 10 is intended to be used to mainly stimulate the external areas of the female genitalia and not for insertion into the vagina. The user determines the speed, the angle, position, and motion at which the active contact portion 12 touches and rubs the clitoris 18. The user can also adjust the amount of pressure applied on the clitoris 18 by adjusting the degree of bend or curve in the device 10. The device 10 is adapted to facilitate stimulation and control using one hand. The active contact portion 12 may be solid or hollow (i.e. tubular) for firmer or softer touch or feel. Optionally, the user may lubricate the active contact portion 12 with a suitable lubricant or lubricating agent and/or the clitoris area for heightened stimulating response and action. Such lubricating agents may include, but not limited to, forms such as water-based lubricants, petroleum-based lubricants, silicones, waxes, oils, jellies, creams, foams, and the like.

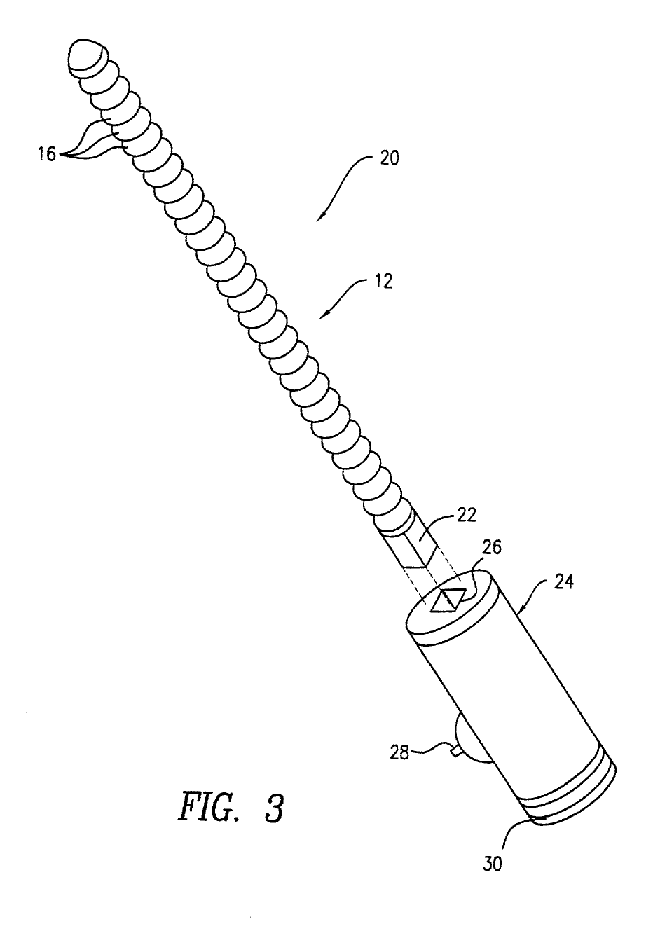

With reference to FIG. 3, a second embodiment of the device 20 is shown according to the present invention. The device 20 includes a projection 22 at the base of the active contact portion 12 which is adapted to connect with a vibrator device 24. The projection 22 snugly fits into a port 26 of the vibrator device 24. The vibrator device 24 generates a vibrating sensation to the active contact portion 12 which is then transmitted to the clitoris 18 for imparting pleasurable tactile sensation thereto. In this embodiment, the vibrator device 24 provides a handle for controlling and manipulating the device 20 by the user. The vibrator device 24 includes a vibrator motor (not shown), a power supply source (not shown), and a vibrator adjustment control (not shown), a rotatable control cap 30. The power supply source, such as a rechargeable battery, is connected to supply power to the vibrator motor causing it to vibrate. The control cap 30 acts to control application of power from the power supply source to the motor. A power switch 28 to be actuated by the user, may be provided for allowing the user to switch on the vibrator motor. Alternatively, the power switch may be incorporated with the rotatable cap 30 component.

The cap 30 is initially turned so that it points to an indicia labeled “OFF”. In this position, the power supply source is separated or disconnected from the vibrator motor. As the cap 30 is turned, the cap 30 causes the power supply source to supply power to the motor. When the power source is turned on, the vibrating motor vibrates and transmits the vibration to the active contact portion 12. The cap 30 is also connected to the vibrator adjustment control whereby when the cap 30 is further turned, the speed of the vibration increases. The speed at which the vibrator motor will vibrate is indicated by the indicia printed around the cap 30. When the cap 30 is turned in the opposing direction the speed of the vibrator motor diminishes until the cap 30 points to the indicia labeled “OFF”. In this position the connection between the power supply source and the vibrator motor is removed.

While a mechanism for producing a vibratory motion causing the active contact portion 12 to vibrate is shown and described herein, those of ordinary skill in the art who have read this description will appreciate that there are numerous other mechanisms for producing a vibratory motion causing the clitoris stimulating device to vibrate, e.g. an oscillator, and, therefore, as used herein the term “vibrator device” should be construed as including all such mechanisms as long as they achieve the desired result of producing a vibratory motion causing the active contact portion 12 of the clitoris stimulating device of the present invention to vibrate, and, therefore, that all such alternative mechanisms are to be considered as equivalents to the one described herein.

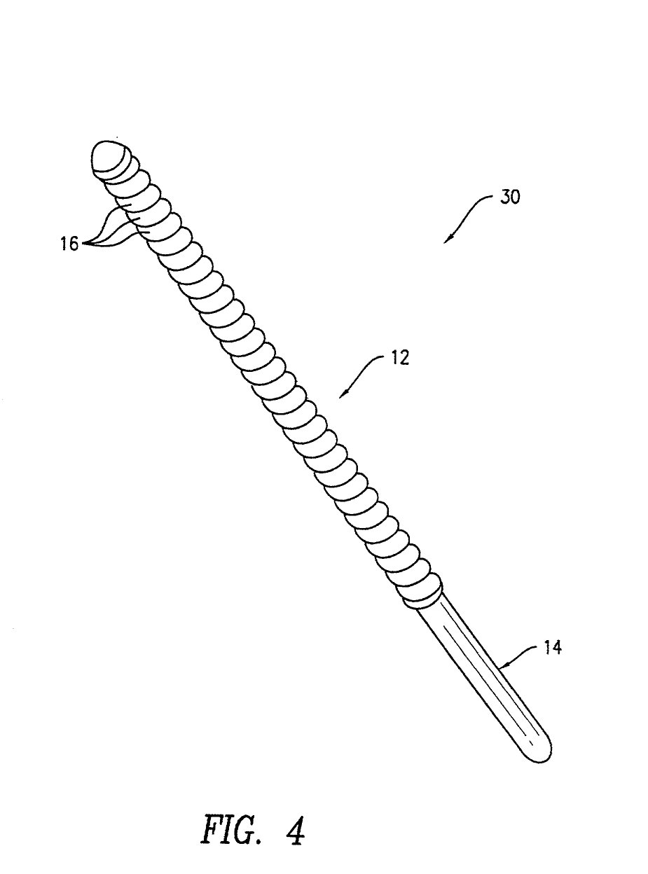

Referring to FIG. 4, a third embodiment of the clitoris stimulating device 30 is shown. The active contact portion 12 includes the plurality of corrugations 16 which are rounded and arranged in a helical or spiral arrangement to illustrate a different form or shape of the corrugations 16. The corrugations 16 provides similar sensations to the user due to the alternating peaks and valleys between each individual corrugation 16. The device 30 is similar in all features to the device 10 except for the surface attributes. The protrusions 34 are in the form of individual raised nubs or droplet shaped.

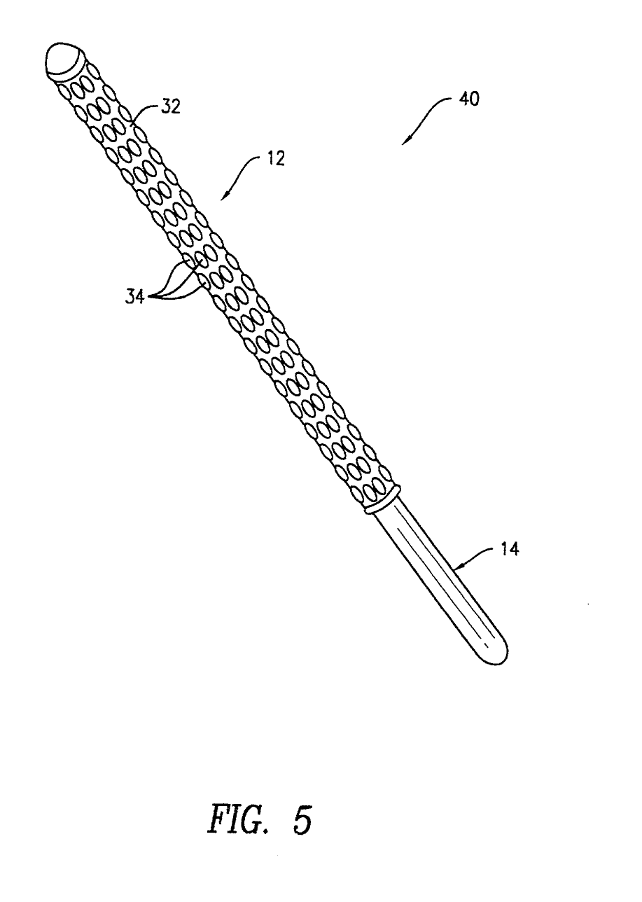

Referring to FIG. 5, a fourth embodiment of the clitoris stimulating device 40 is shown. The active contact portion 12 comprises a smooth surface 32 with a plurality of protrusions 34 distributed thereacross. The device 40 is similar in all features to the device 10 except for the surface attributes.

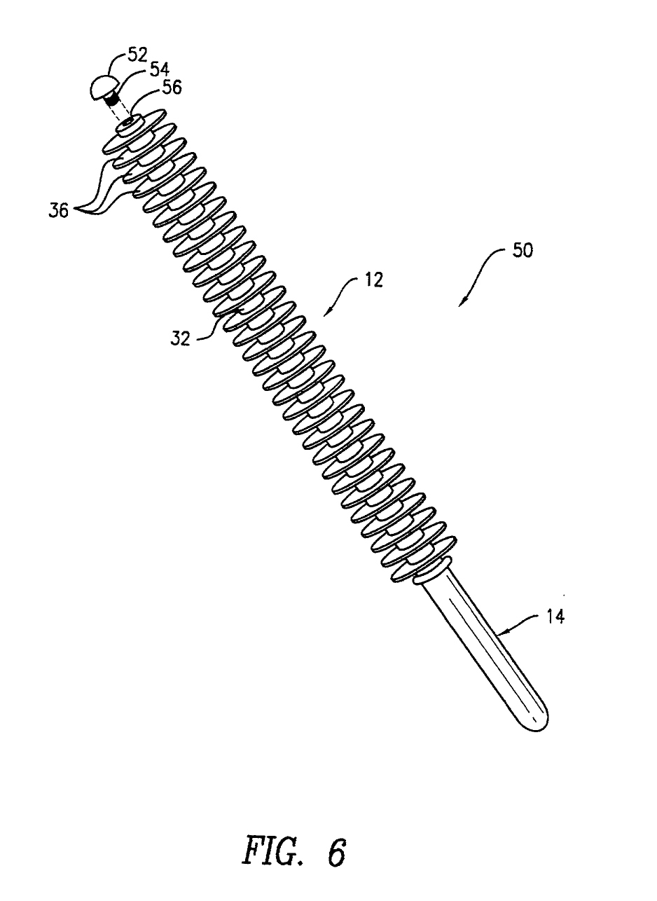

Referring to FIG. 6, a fifth embodiment of the clitoris stimulating device 50 is shown. The active contact portion 12 includes a plurality of ridges 36 arranged in a spaced apart manner along the surface 32 of the device 50. The ridges 36 are preferably composed of a flexible material such as an elastomer or similar material. The elastomer material may be silicone rubber or polyurethane which is resilient, durable, and hypoallergenic. The device 50 is used in the same manner as described above. The device 50 is similar in all features to the device 20 except for the surface attributes.

The present invention may further include a feature whereby liquid may be retained within the interior of the device. The liquid may be heated or cooled to a specific temperature within the device of the present invention for heightening the tactile feeling or sensation during use. In one embodiment as shown in FIG. 6, the device 50 may optionally include a cap 52 with retention threads 54 adapted for threaded engagement with a hole 56 located at the tip thereof. The device 50 includes an interior cavity (not shown) which is configured for holding a liquid of a specific temperature. The device is filled with the liquid through the hole 56 and the cap 52 securely screwed into the hole 56 for sealing the liquid. The temperature of liquid in the device 50 may be varied according to the user's desires.

The operation of the clitoris stimulating device 10 of the present invention will now be described with reference to FIGS. 1, 2, and 3. In operation, the device 10 is held by the user at the handle 14 in a comfortable position. The active contact portion 12 is positioned next to the clitoris 18 of the user. The device 10 is moved back and forth in a manner similar to a bow moving back and forth across the strings of a stringed musical instrument. The user may adjust the angle, speed and the pressure of the device 10 on the clitoris 18. The active contact portion 12 rubs against the clitoris 18 until sexual arousal and orgasm is achieved. Optionally, in the embodiment utilizing a vibrator device 24, the user may grasp and turn the cap 30 to an ON position and adjusted to a desired vibration speed. At this point, the vibrator motor is turned on where it stimulates the clitoris 18. The user now begins sexual activity. Once the user has completed the sexual activity, the cap 30 is turned in the opposite direction to an OFF position.

The clitoris stimulating device of the present invention may be formed of any type of material which will perform the above described functions and will not react adversely with the skin of a user such as to cause chafing, rashes, rawness, inflammation, or any other reaction.

Although various embodiments of the invention have been shown and described, they are not meant to be limiting. Those of skill in the art may recognize various modifications to these embodiments, which modifications are meant to be covered by the spirit and scope of the appended claims. For example, each embodiment of the invention can be provided by molding or extruding the associated material.

What is claimed is:

1. A clitoris stimulating device comprising:
an elongate member including proximal and distal ends; and
a plurality of corrugations extending in successive arrangement therebetween.

2. The device of claim 1, wherein each of said plurality of corrugations comprises a plurality of successive smooth, curvilinear surfaces.

3. The device of claim 2, wherein each of said plurality of corrugations comprises a disk-like radial projection.

4. The device of claim 1, wherein the elongate member is composed of a semi-rigid material.

5. The device of claim 4, wherein the semi-rigid material is selected from the group consisting of plastic polymers, and elastomers.

6. The device of claim 1, wherein the proximal end further includes a handle.

7. The device of claim 6, wherein the handle is further adapted for operative engagement with a vibrator device.

8. The device of claim 1, wherein the elongate member is tubular.

9. The device of claim 5 wherein the plastic polymer is nylon.

10. The device of claim 1, wherein each of the plurality of corrugations comprises a flexible ribbing.

11. The device of claim 4, wherein the semi-rigid material is selected from the group consisting of silicone rubber, and polyurethane.

12. The device of claim 1, further comprising a vibrator device located at the proximal end of the elongate member.

13. The device of claim 1, wherein the plurality of corrugations comprise a curvilinear spiral.

14. The device of claim 1, wherein the plurality of corrugations comprise successively stacked juxtaposed rings.

15. A clitoris stimulating device, comprising:
an elongate sleeve member having proximal and distal ends;
a plurality of ridges extending between said proximal and distal ends; and
a handle located at the proximal end of the sleeve.

16. The device of claim 15, wherein the plurality of ridges comprise a plurality of protuberances disposed on the surface of the sleeve member.

17. The device of claim 15, wherein the sleeve is comprised of a semi-rigid material.

18. The device of claim 17, wherein the semi-rigid material is selected from the group consisting of plastic polymers, and elastomers.

19. The device of claim 15, wherein the handle is rigid.

20. The device of claim 15, wherein the elongate sleeve is adapted to bend into a curvilinear shape.

21. The device of claim 15, wherein said plurality of ridges are composed of a flexible, elastic material.

22. A clitoris stimulating device, comprising:
an elongate member including a plurality of radially directed projections arranged in abutting engagement for forming a series of serrations;
said elongate member being composed of a semi-rigid material; and
a vibrator device connected to said elongate member for generating vibrations longitudinally along the elongate member to the each of the plurality of projections.
